(19) United States
(12) Patent Application Publication
(10) Pub. No.: US 2017/0043370 A1
(43) Pub. Date: Feb. 16, 2017

(54) TEXTURED CONDOM

(71) Applicant: Ansell Limited, Richmond (AU)

(72) Inventors: KC NGUYEN, Dothan, AL (US); Chayaporn Pongthanomsak, A. Phunphin Surat Thani (TH); Beng Sim Chuah, Petaling Jaya (MY)

(21) Appl. No.: 15/339,836

(22) Filed: Oct. 31, 2016

Related U.S. Application Data
(62) Division of application No. 13/928,631, filed on Jun. 27, 2013.
(60) Provisional application No. 61/667,770, filed on Jul. 3, 2012.

(51) Int. Cl. B05D 1/28; B05C 1/02; A61F 6/04

(52) U.S. Cl. CPC B05D 1/28; A61F 6/04; B05C 1/022; A61F 2006/048

(57) ABSTRACT

A condom including a polymeric sheath, and one or more features including a polymeric composition disposed thereon, the one or more feature optionally having a color different than that of the polymeric sheath, is disclosed. Apparatus and methods for manufacturing a textured condom are also disclosed.

### CROSS-REFERENCE TO RELATED APPLICATIONS

This application is a divisional of co-pending U.S. patent application Ser. No. 13/928,631 filed on Jun. 27, 2013 which claims the benefit of U.S. provisional patent application Ser. No. 61/667,770, filed Jul. 3, 2012, which is hereby incorporated by reference in its entirety.

### BACKGROUND

Field of the invention

Embodiments of the present invention generally relate to condoms and, more particularly, to condoms comprising one or more textures, and methods of fabricating such condoms.

Description of the Related Art

Polymeric articles such as condoms and other prophylactics and protective devices provide physical barriers against the transmission of bodily and other fluids. Typical condoms include a generally tubular polymeric sheath having an open end and a closed end. The closed end generally tapers to close the otherwise tubular shape of the condom, and in general, the condom is formed to match the shape of a penis. Designers have further attempted to create condoms having textures, patterns, and other adornments to provide different visual appearances as well as features promoting physical stimulation. For example, condoms have raised studs or ribs, which are the negative of a condom former. Furthermore, some condoms have studs or ribs that are embossed onto the surface of the condom. Other condoms are created using a condom former having studs or rib structures etched into the former surface. However, condoms manufactured using such processes require additional manufacturing steps, additional manufacturing time, are expensive, and are limited in design choices.

Therefore, there is a need in the art for a condom having various textures comprising, for example, different colors or patterns, or designs disposed on the condom, without requiring the use of textured formers or embossing operations.

### SUMMARY

Embodiments of the invention include polymeric articles, such as a polymeric sheath having various textures disposed thereon to form condoms, and a method for manufacturing textured polymeric articles, substantially as shown in and/or described in connection with at least one of the figures are disclosed herein, as set forth more completely in the claims. Various advantages, aspects, and features of the present disclosure, as well as details of an illustrated embodiment thereof, will be more fully understood from the following description and drawings.

### BRIEF DESCRIPTION OF THE DRAWINGS

So that the manner in which the above recited features of the present invention can be understood in detail, a more particular description of the invention, briefly summarized above, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments. It is to be understood that elements and features of one embodiment may be in other embodiments without further recitation. It is further understood that, where possible, identical reference numerals have been used to indicate comparable elements that are common to the figures.

FIG. 1A depicts an apparatus for manufacturing condoms in accordance with embodiments of the invention;

FIGS. 1B-1E depict an apparatus for manufacturing condoms in accordance with embodiments of the invention;

FIG. 2 depicts a partial view of a roller and a polymeric sheath disposed on a former in accordance with embodiments of the invention;

FIG. 3 depicts a partial view of a roller and a polymeric sheath disposed on a former in accordance with embodiments of the invention;

FIG. 4 depicts a condom according to embodiments of the invention;

FIG. 5 depicts a cross-sectional view of the condom depicted in FIG. 4, taken along line 5-5, in accordance with embodiments of the invention; and

FIG. 6 depicts a flow diagram of a method for manufacturing a condom in accordance with one or more embodiments of the invention.

### DETAILED DESCRIPTION

Embodiments in accordance with the present invention pertain to a polymeric article, such as a condom, comprising a polymeric sheath having textures, such as ribs, protrusions, stripes, and/or other patterns and features made of polymeric material disposed thereon to form a textured condom. In some embodiments, the condom has one or more features which may be colored and have a color similar or different than that of the polymeric sheath. Textures, features, and patterns may be formed on any portion of the condom surface and comprise different colors.

Embodiments in accordance with the invention comprise various textures on condoms irrespective of the former used in manufacturing the condom. The former generally conforms to the shape of a penis, and may have additional textures or shape variations thereon. Textures comprise the deposition of polymeric material onto an external surface of a polymeric sheath and include features having raised dimensions (ribs, protrusions, stripes, rings, and the like), which include logos, shapes, graphics, symbols, alphabetic, or numeral characters, and the like. The textures may comprise features visibly raised from the external surface of the polymeric sheath, which can also impart physical and/or visual stimulation, and/or appear imperceptibly raised from the surface of the polymeric sheath.

As used herein, the terms “pattern,” “texture,” and “feature” are used to describe material, such as a polymeric composition, applied on the external surface of the polymeric sheath to form condoms in accordance with embodiments of the invention. The terms “patterns,” “textures,” and “features” may be used interchangeably herein. In some embodiments, features include a coagulant composition deposited on a polymeric sheath, and a polymeric composition deposited over the coagulant composition. According to some embodiments, features include one or more of coagulant compositions and polymeric compositions deposited in one or more layers onto a polymeric sheath. Each feature optionally comprises one or more colors. Polymeric compositions include elastomeric compositions, latex compositions, and rubber compositions and/or blends or mixtures thereof. According to some embodiments, a former is dipped into a bath of polymeric composition to form a polymeric sheath over the former. Additional textures or features (patterns such as ribs, protrusions, or stripes and the like) of comprising a polymeric composition, and/or a coagulant composition are subsequently deposited on the external surface of the polymeric sheath without further dipping processes, allowing features to be formed on the polymeric sheath, as discussed more fully below, to form a condom.

In one or more embodiments, the condom is formed from a polymeric composition comprising one or more of synthetic rubber, natural rubber latex, thermoplastic elastomer, or combinations, mixtures, or blends thereof. Other examples of suitable polymeric compositions include synthetic polyisoprene, guayule, polyurethane, polyethylene, copolymers, block copolymers, and blends, mixtures, or combinations thereof. The term “natural rubber latex” as used in this disclosure encompasses cured elastomeric material sourced from Hevea brasiliensis (the traditional rubber tree), Parthenium argentatum (guayule), sunflower, goldenrod, and the like, as well as genetically modified variations of these or other biological sources. In some embodiments of the invention, condoms comprise the pre-vulcanized and post-vulcanized latex composition as disclosed in commonly-assigned U.S. Pat. No. 8,087,412, which is hereby incorporated by reference in its entirety.

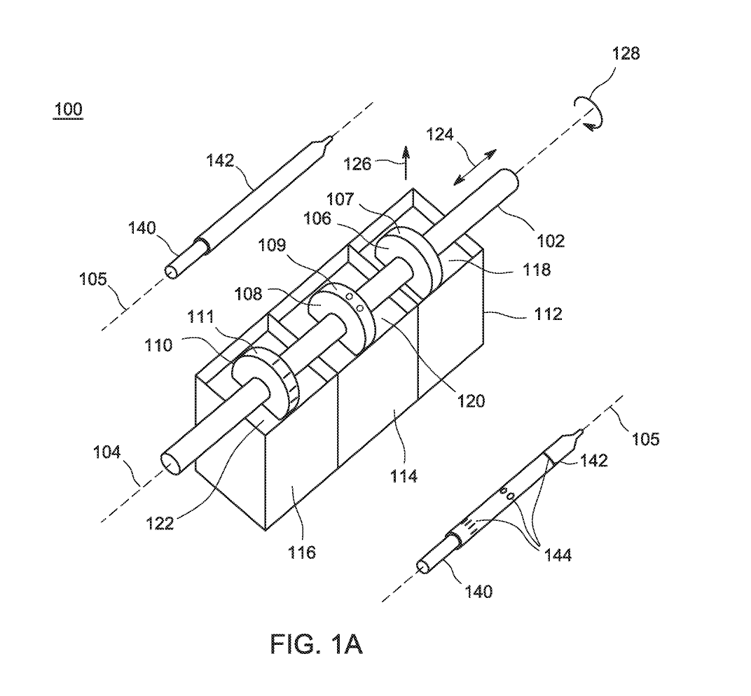

FIG. 1A depicts an apparatus 100 for manufacturing condoms in accordance with embodiments of the invention. The apparatus 100 comprises a shaft 102 with multiple rollers 106, 108, 110 disposed thereon, configured to rotate with the shaft 102. The rollers 106, 108, and 110 comprise surfaces 107, 109, and 111, respectively. In some embodiments, the apparatus 100 comprises a single roller, and in other embodiments the apparatus 100 comprises multiple rollers. According to some embodiments, the rollers 106, 108, 110 are made of rubber and/or other elastomers, for example, natural or synthetic rubbers, such as polyisoprenes, styrene-butadiene, nitriles, thermoplastics, or metals, such as alloys, for example, steel, brass, and the like, or combinations thereof. The rollers 106, 108, 110 are configured to dip partially within multiple tanks 112, 114, 116, respectively. The tanks 112, 114, 116, each comprise a coating solution 118, 120, 122, respectively.

The coating solutions 118, 120, and 122 include one or more of a polymeric composition, coloring agents, pigments, and other processing aids, additives, rheological additives, and the like known to those in the art. The polymeric composition is similar to the polymeric composition discussed above, and in some embodiments, one or more of the coating solutions 118, 120, and 122 comprise a latex emulsion. The total solids content range of the latex emulsion, which may include a natural color or another color, may range from about 40% to about 70%. In some embodiments of the invention, total solids content range of the latex emulsion may be about 53%. In some embodiments, the coagulant concentration ranges from about 1% to about 50% by weight, and may include a natural color or another color. In some embodiments of the present invention, the coagulant concentration is about 5% by weight. According to some embodiments, the coagulant solution may contain Group I metal salts, Group II metal salts, or combinations thereof, and wetting agents ranging from 0.1-0.2% by weight in an aqueous solution. In some embodiments of the invention, the coagulant is an aqueous solution comprising 3.5% calcium nitrate and 96.5% water. Other suitable coagulants known to those in the art may also be used. In some embodiments of the invention, one or more of the tanks 112, 114, 116 comprise a coating solution having at least one color different than that of the polymeric sheath 142 on which the coating solution is deposited.

Coating solutions including polymeric compositions, such as latex are provided in desired colors, for example, according to the techniques as known to one of ordinary skill in the art, using various pigments, dyes, inks, and the like. Suitable pigments used in embodiments in accordance with the invention comprise any pigment or colorant compatible with polymeric, elastomeric, rubber, latex, and like materials. In some embodiments, pigments supplied by the Clariant Corp., such as Colanyl® Red E3B500, Flexonyl® Yellow DRG LA and Akrosperse® colors E1399 Blue, E1180 Violet, E2547 Green, are used. Any combination of any of these colors can be used to make other colors. The concentration of the pigments, whether in the polymeric composition or the coagulant solution, ranges from about 0.05% to about 2.0%. In some embodiments, the concentration of the pigments is approximately 1%.

FIG. 1A further depicts a former 140 on which a polymeric sheath 142 is disposed thereon before being processed by the apparatus 100. The polymeric sheath 142 may be formed or dressed thereon. The external surface of the polymeric sheath is exposed while an internal surface is adjacent to a former. Upon processing by the apparatus 100, the polymeric sheath 142 has one or more features 144 deposited thereon the external surface of the polymeric sheath using the apparatus 100, for example, as discussed further below.

The rollers 106, 108, and 110 are partially immersed within the coating solutions 118, 120, and 122. The shaft 102 and the rollers 106, 108, 110, are rotatable about its longitudinal axis 104, for example, in the direction denoted by numeral 128. The rotation of the rollers causes at least some composition from the tanks to adhere to at least a part of the surface 107, 109, and 111 of the rollers, as discussed below. Accordingly, the rollers 106, 108, 110 are charged with the coating solution 118, 120, 122 from the tanks 112, 114, 116, respectively, and at least some of the coating solution adhered to the surface 107, 109, 111 of the rollers is transferred, by means of physical contact, to an external surface of the polymeric sheath 142 disposed on former 140. The former is rotatable about its longitudinal axis 105. The duration of the physical contact between the polymeric sheath 142 and the one or more rollers 106, 108, 110 while the polymeric sheath 142 contacts the respective surfaces 107, 109, 111 determines the amount of the coating solution deposited on the polymeric sheath 142. Also, additional rotations can impart a thicker layer of a coating solution onto the polymeric sheath 142.

In some embodiments of the invention, the shaft 102 and the rollers 106, 108, and 110 are also movable along the longitudinal axis 104, as denoted by numeral 124. In several embodiments, the rollers 106, 108, 110 move along the longitudinal axis 104 with respect to the polymeric sheath 142, while the rollers 106, 108 and 110 are submerged in the coating solutions 118, 120 and 122 respectively (e.g., within a particular tank). According to some embodiments, the relative longitudinal movement between the rollers 106, 108, 110 and the polymeric sheath 142 allows the disposition of coating solution onto the polymeric sheath 142, for example, over a larger section thereof, and/or in patterns formed due to the relative motion thereof, as discussed below. Further, rollers may be moved across various tanks and deposit different coating solutions onto a polymeric sheath.

FIG. 1B-1E depicts an apparatus 100 for manufacturing condoms in accordance with embodiments of the invention. As illustrated in FIG. 1B, the shaft 102 has the rollers 106, 108, 110 disposed thereon. The rollers 106, 108, 110 are partially dipped in tanks 112, 114, 116, comprising coating solutions 118, 120, 122, respectively. According to embodiments of the invention, the shaft 102 is moved to remove the rollers 106, 108, 110 from the tanks 112, 114, 116 such that the rollers 106, 108, 110 can move across the tanks in which the rollers were previously dipped. For example, as illustrated by FIG. 10, the shaft 102 is moved to remove the rollers 106, 108, 110 completely out of the tanks 112, 114, 116 such that a longitudinal movement of the shaft 102 along the axis 104 is not blocked by a tank boundary. As illustrated by FIG. 1D, the shaft 102 (along with the rollers 106, 108, 110) is moved along the axis 104 such that the rollers 106, 108, 110 are shifted to be above the adjacent tanks. That is, the roller 110, which was previous dipped in the tank 116, is shifted to be above the tank 114, the roller 108 is shifted above the tank 112, and the roller 106 is shifted outside the tanks 112, 114, 116, as shown. As illustrated by FIG. 1E, the shaft 102, and therefore the rollers 106, 108, 110, are lowered such that the rollers 110 and 108 are dipped in the tanks 114 and 112, respectively, no roller is dipped in the tank 116, and the roller 106 is not dipped into any of the tanks 112, 114, 116. The rollers 110 and 108 may then be re-engaged with the polymeric sheath 142 to deposit the coating solutions 120 and 118 respectively on the polymeric sheath 142, for example, as discussed above. As shown, the roller 106 is not charged with any coating solution and accordingly does not deposit any material on the polymeric sheath 142.

In this manner, one roller is usable to deposit multiple coating solutions from multiple tanks, onto a polymeric sheath. For example, the roller 110 deposits the coating solutions 122 and 120 from the tanks 116 and 114, respectively, onto a polymeric sheath.

According to embodiments of the invention, the shaft 102 (and therefore the rollers 106, 108, 110 disposed thereon) and/or the former 140 on which the polymeric sheath 142 is disposed, are controlled by a motive force from an arrangement (not shown) comprising one or more motors, gears, transmissions, or equivalent mechanisms known in the art. Such mechanisms provide for positioning, imparting motion, such as rotational or linear motion, to the former and/or the rollers. According to some embodiments, the shaft 102 is rotatable about the axis 104. According to some embodiments, the shaft 102 is movable along the axis 104 and/or perpendicular direction to the axis 104, as denoted by numerals 124 and 126, respectively. According to some embodiments, the former 140 is rotatable along its longitudinal axis 105, and optionally movable along the axis 105 and/or perpendicular to the axis 105. At least one of the former 140 and the shaft 102 are movable to engage the polymeric sheath 142 with the surfaces 107, 109, 111 of the rollers. In some embodiments, the rotation of the rollers 106, 108, 110 causes rotation of the polymeric sheath 142. In some embodiments, the rotation of the former 140, on which a polymeric sheath may be disposed or other dressed thereon, causes rotation of the rollers 106, 108, 110. In some embodiments, the rotations of the former 140 and the rollers 106, 108, 110 are independently controlled and counter-rotate.

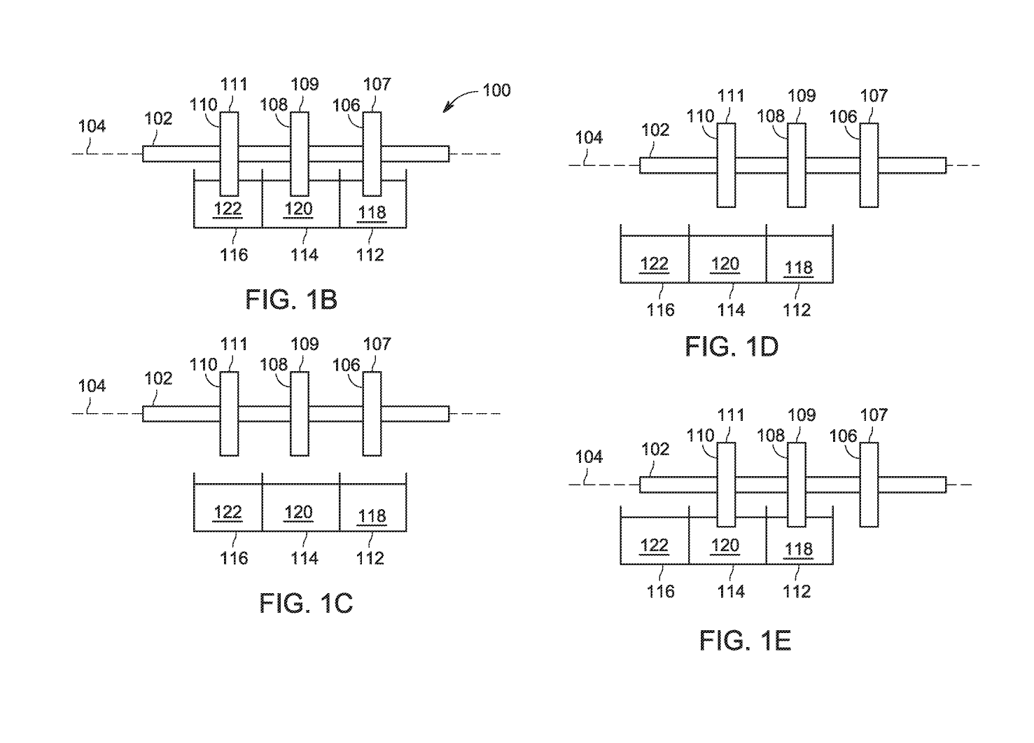

FIG. 2 depicts a partial view 200 of a roller 210 and a polymeric sheath 230 disposed on a former in accordance with embodiments of the invention. The roller 210 is, for example, similar to the rollers discussed above. The polymeric sheath 230 is similar to the polymeric sheath on the former as discussed above. The roller 210 and the polymeric sheath 230 are rotatable about respective axes 212 and 232. The roller 210 rotates in, for example, a direction 214, while the polymeric sheath 230 rotates in a counter direction 234. In operation, the roller 210 and polymeric sheath 230 are in physical contact and generally rotate in opposite directions.

The roller 210 comprises one or more templates on a surface 211 of the roller 210, as denoted by numerals 216, 218, and 220. For example, the templates 216, 218, and 220 are configured according to patterns or features desired to be deposited on an external surface of the polymeric sheath 230. In some embodiments, a roller has a plain surface, and has no feature on the surface of the roller. The plain surface of a roller in such embodiments (not shown) acts as a pattern to deposit polymeric composition, for example, in a texture or pattern resembling a line, band, stripe, or a portion thereof. Upon rotation, at least a part of the roller 210 including the templates 216, 218, and 220, submerges in polymeric composition, for example, the polymeric composition discussed above. The polymeric composition adheres to one or more of the templates 216, 218, and 220. Upon further rotation, the roller 210 deposits the polymeric composition onto the polymeric sheath 230 upon contact, as features 236, 238, and 240, which are mirror images of the templates 216, 218, and 220. As it rotates, the roller 210 is continually recharged with coating solution, for example, from the respective tank. As the roller 210 continues to rotate, additional polymeric composition is disposed onto the polymeric sheath 230.

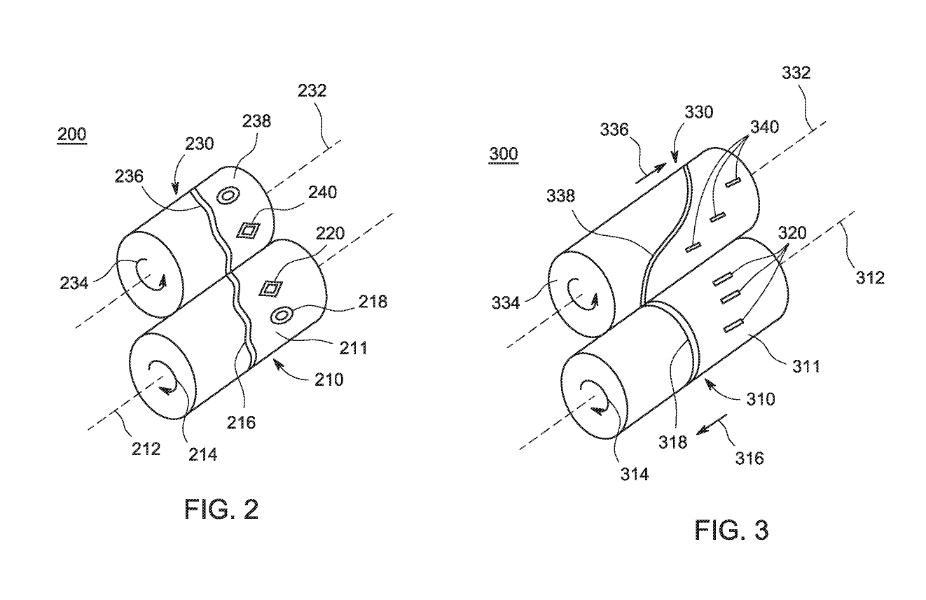

FIG. 3 depicts a partial view 300 of a roller 310 and a polymeric sheath 330 disposed on a former in accordance with embodiments of the invention. The roller 310 is rotatable about an axis 312, in direction 314, and the polymeric sheath 330 is rotatable about an axis 332 in a direction 334. The roller 310 and the polymeric sheath 330 are in physical contact, and rotate in opposite directions, similar to the roller and the polymeric sheath discussed above. In some embodiments, the roller 310, or the polymeric sheath 330 disposed thereon, or both, are also movable in a longitudinal relative direction, for example, as illustrated by numerals 316 or 336. The roller 310 has templates 318, 320 disposed on a roller surface 311, as illustrated, and by virtue of rotation as well as longitudinal relative motion, the roller 310 deposits the features 338 and 340 respectively on the polymeric sheath 330. The combination of relative rotational and relative longitudinal motion between the roller 310 and the polymeric sheath 330 determines the translation of the templates 318, 320 of the roller 310 to the features 338, 340 deposited on polymeric sheath 330. According to some embodiments, the relative motion between a condom disposed on a former and the roller(s), is modulated to dispose features having a wavy or a sinusoidal pattern.

Additionally, according to embodiments of the invention, one or more rollers may be dipped in different polymeric compositions, configured to deposit features, comprising the different polymeric compositions, in distinctive colors, for example. According to some embodiments of the invention, the polymeric sheath is dipped in coagulant material before deposition of the features, after deposition of the features, or both. The application of coagulant material destabilizes the polymeric composition, allowing an increase in the amount of polymeric composition disposed on the polymeric sheath in a single pass of a roller, and in some cases increases the strength of bonding between polymeric material disposed by a roller on polymeric sheath and the polymeric sheath. However, according to some embodiments, coagulant material in not deposited on a polymeric sheath prior to disposing the desired features thereon.

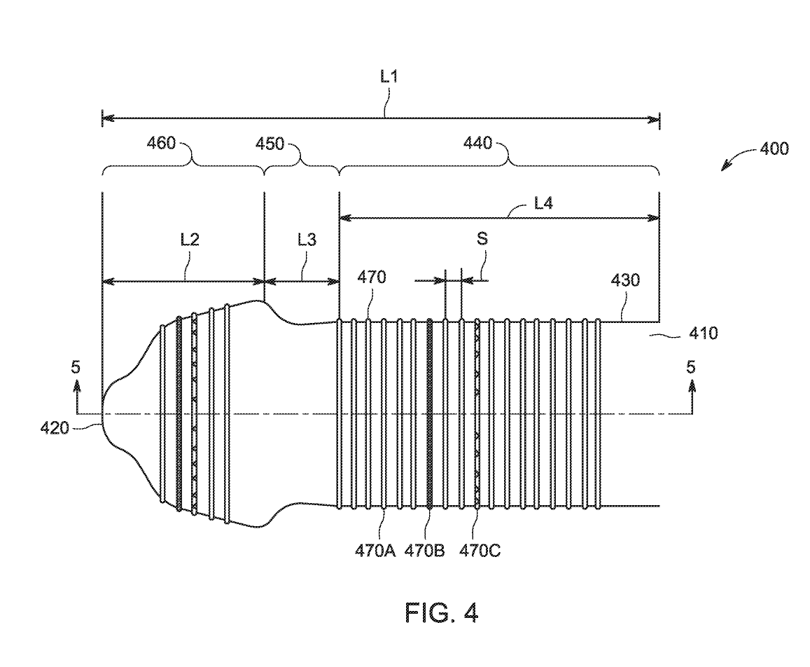

FIG. 4 depicts a condom 400 according to embodiments of the invention. The condom 400 may be manufactured using one or more rollers having templates including single or multiple stripes or ring patterns on the roller surface, and depositing coating material such as a polymeric composition on an external surface of a polymeric sheath 430, similarly as discussed above. As illustrated by FIG. 4, multiple features, such as rings 470, are deposited on the condom 400. By way of example, and not limitation, the rings 470 comprise a polymeric composition, such as latex, to form a feature, texture, or pattern deposited on the polymeric sheath to form condom 400, in a manner similar to the feature formation as discussed above. The polymeric sheath 430 has a length L1, and comprises an open end 410, and a closed end 420. The condom 400 includes a base portion 440 (length L4) disposed adjacent to the open end 410. The polymeric sheath 430 also includes a middle portion 450 (length L3) extending from the base portion 440 toward a tip portion 460 (length L2). In certain embodiments, a part of the middle portion 450 adjacent to the tip portion 460 is adapted to match, approximately, the retroglandular sulcus of a wearer's penis.

The rings 470A, 470B, 470C are circumferential, and traverse the entire circumference of the condom 400 surface, forming ring structures around the condom 400. Embodiments according to the invention include the ring 470A having the same color as the polymeric sheath 430, while the rings 470B and 470C have a different color than the polymeric sheath 430. In practice, any ring, or other feature, can have any color and any combination of differently colored feature is possible. Also, in some embodiments, the rings 470 are spaced at a uniform distance denoted by S, though, in some embodiments, distance S may vary, as discussed below.

According to embodiments of the invention, the condom 400 has at least one feature, for example, a ring 470B having a color different than that of the polymeric sheath 430, thereby resulting in a condom with multi-color features. In some embodiments, multiple rings have a color that is different from the color of the polymeric sheath. The features include a stripe (not shown) disposed substantially along the length L1 of the polymeric sheath 430 or condom 400. In embodiments having several rings 470, the rings 470A, 470B, 470C may comprise the same or a different color from each other.

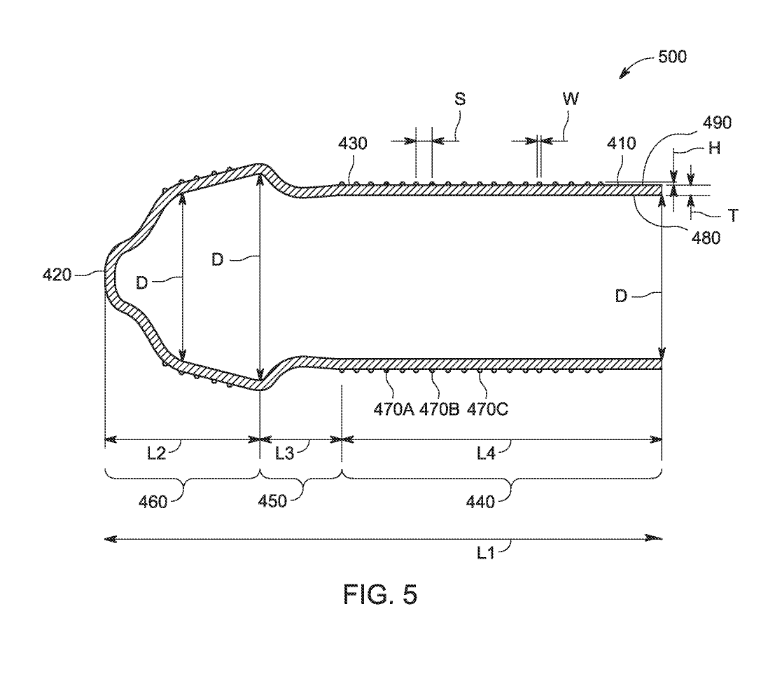

FIG. 5 depicts a cross-sectional view 500 of the condom 400 depicted in FIG. 4, taken along line 5-5, in accordance with embodiments of the invention. The cross section depicts the polymeric sheath 430 having an internal surface 480 and an external surface 490, and features 470 such as the rings 470 disposed on the polymeric sheath 430. The base portion 440 and the middle portion 450 have a combined length (L3+L4) in the range from about 95 mm to about 145 mm. Alternatively, the base portion 440 and the middle portion 450 have a combined length (L3+L4) of about 120 mm. In one or more embodiments, the base portion 440 has a length L4 that is greater than the length L3 of the middle portion 450. In one or more embodiments, the base portion 440 length L4 is in the range from about 90 mm to about 120 mm. In some embodiments, the length L4 of the base portion 440 is about 105 mm. According to several embodiments, the length L3 of the middle portion 450 is varied, for example, according to desired specifications for the condom 400. In one or more embodiments, the polymeric sheath 430 does not include a middle portion, and includes only the base portion 440 and the tip portion 460. The tip portion 460, has a shape adapted to fit over the glans of a penis, and has a length L2, for example, in the range from about 50 mm to about 70 mm. In some embodiments, the tip portion 460 length L2 is in the range from about 55 mm to about 65 mm. In some embodiments, the length L2 is about 60 mm. The overall length L1 of the condom 400, which is a sum of the lengths L2, L3, and L4, is between about 120 mm to about 215 mm.

In one or more embodiments of the invention, the average cross-sectional thickness T of the polymeric sheath 430 is approximately 0.05 mm. In some embodiments, the polymeric sheath near the open end 410 of the condom 400 has a cross-sectional thickness T ranging from about 0.05 mm to about 0.10 mm. The cross-sectional thickness T of polymeric sheath 430 may, optionally, decrease along the base portion 440 and the middle portion 450 from the open end 410 to the closed end 420, providing a thinner condom to impart additional sensitivity near the retroglandular sulcus. In one or more embodiments, the cross-sectional thickness T of the polymeric sheath 430 decreases substantially linearly from the open end 410 to the closed end 420.

In at least one embodiment of the invention, one or more portions (tip, middle portion, base, and the like) of the polymeric sheath 430 do not include a feature deposited thereon. For example, the rings 470 are disposed on the base portion 440 and the tip portion 460, but not disposed on the middle portion 450. According to several embodiments, however, any combination of the base portion, the middle portion, and the tip portion include one or more patterns or features. The rings 470 may be separated uniformly by space S as discussed above with respect to FIGS. 4 and 5, however, in other embodiments, space S may be different across any two adjacent rings 470, to create different visual appearances, create varied physical stimulation, or both. According to several embodiments, the space S ranges from about 0.5 mm to about 50 mm. In some embodiments, the space S is about 4 mm, and in some embodiments, the space S is about 2 mm. In some embodiments, the rings 470 are disposed at regular intervals, or at irregular intervals, in some or all portions of the condom 400.

Further, the rings 470 have a height H, as illustrated in the cross section 500, extending outwards from the polymeric sheath 430. In some embodiments, the height H of the rings 470 ranges from about 0.01 mm to about 2 mm, in some embodiments, the height H is about 0.4 mm, and in some embodiments, the height H is more than 2 mm. The height H of rings 470 is increased by depositing more coating solution onto the polymeric sheath 430.

According to embodiments of the invention, the diameter D of the condom 400 varies along the length L1 of the condom 400. According to several embodiments, the diameter D varies from about 4 cm to about 6 cm within the tip, middle, and base portions of the condom 400.

Features disposed on the condom have been illustrated, by way of example, with the rings 470, and according to several embodiments, the condom 400 is disposed with other features including ribs, protrusions, incomplete rings, spiral formations, dotted lines, features in circular shape, diamond shape, sinusoidal shapes, and several other shapes that include one or more of visually appealing, or physical stimulation enhancing shapes, or a combination thereof. Furthermore, according to embodiments of the invention, one or more of the features has at least one color different from that of a polymeric sheath that the features are disposed on. The features may be selected with different dimensions, for example, dimensions comprising height H, width W and space S between adjacent features. In some embodiments, the condom 400 is larger or smaller, and it will be understood that the remaining portions of the condom may also be larger or smaller in a proportional manner, as known to those having ordinary skill in the art.

Methods for forming condoms in accordance with embodiments of the present invention are also included herein. In one or more embodiments, at least one method includes providing a former comprising an axial length, a circumference, and a plurality of depressions, ribs, or protrusions disposed along at least a portion of the length and around or along the circumference of the former as is disclosed in commonly assigned U.S. provisional application Mo. 61/385,694, filed Sep. 23, 2010; and U.S. patent application Ser. No. 13/243,038, filed Sep. 23, 2011, each of which is hereby incorporated by reference in its entirety. The method of one or more embodiments includes disposing a polymeric layer on the former, generally by a dipping process, to form a polymeric layer (or sheath) on the former, as otherwise described herein, and optionally curing the polymeric sheath to form a cured polymeric sheath.

The former may be a smooth former or, alternatively, a former having depressions on the surface, which create ribs on the inside surface of a condom. In one or more embodiments, the former may include a tubular body having a first end and a second end. The tubular body may have an overall shape that is similar to the shape of a penis, thereby resulting in the polymeric sheath of the condom described above. The tubular body of the former may include a base segment that is disposed adjacent to the first end and extends from the first end toward the second end. In one or more embodiments, the second end is utilized to form a closed end of the condom described above, while the first end of the former is utilized to form an open end and a base portion of the condom described above. The tubular body of the former according to one or more embodiments may also include a middle portion that extends from the base portion toward a tip portion, which is disposed adjacent to the second end.

In one or more embodiments, the step of disposing a polymeric sheath on the former includes disposing a coagulant component on the former, as is known to those of skill in the art, and dipping the coagulant coated former into a bath or tank containing a polymeric composition as described herein. In one or more embodiments, the former may be dipped in a bath or tank containing a polymeric composition without first disposing a coagulant component on the former. Other methods of disposing a polymeric layer on the former may be utilized, such as spraying or solvent dipping. Additionally, the temperature of the polymeric composition may be controlled, as is known in the art, and may include additives to control or modify the properties of the elastomeric composition, such as the viscosity of the composition as well as the physical properties, for example, lubricity, tensile strength, puncture resistance, and the like, of condoms formed therefrom.

The polymeric composition of one or more embodiments may also include a cure package or vulcanization agents to promote cross-linking during the curing process. As the former is dipped into a bath or tank, the dwell time and immersion and extraction speeds of the dipping process may be controlled and modified to adjust the thickness of the resulting layer that forms the polymeric sheath. In one or more embodiments, the polymeric composition disposed on the former is cured or otherwise treated to form a cured polymeric layer. In one or more embodiments, the polymeric layer is dried in ambient air and heated to a temperature in the range from about 50° C. to about 150° C. In one or more embodiments, the method utilizes a former that includes a plurality of depressions disposed on the surface thereof to form ribs or protrusions on embodiments of the condom described above. The former may be formed from a ceramic material, metallic material, or other material known in the art.

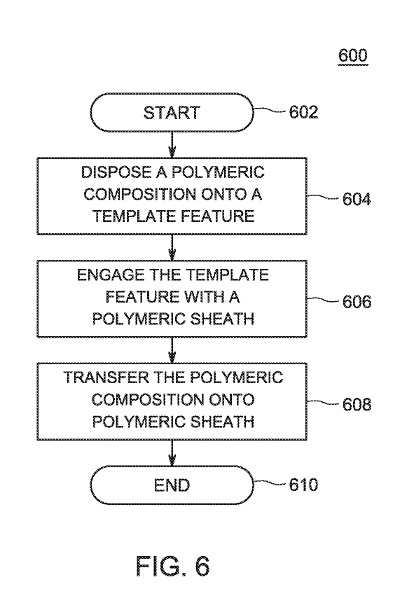

FIG. 6 depicts a flow diagram of a method 600 for depositing multiple features on a polymeric article, for example, a condom, in accordance with one or more embodiments of the invention. In some embodiments, each and every step of the method 600 is performed. In some embodiments, some steps are omitted or skipped or additional steps are performed. Also, in some embodiments, steps may be implemented in different sequences.

In at least one embodiment of the invention, method 600 starts at step 602 and proceeds to step 604, at which point a polymeric composition is disposed onto a template, for example, the templates disclosed herein with respect to the apparatus for manufacturing condoms. According to embodiments of the invention, the templates are disposed on one or more rollers as discussed above. The polymeric composition is configured for appropriate concentration of polymeric material(s) and optionally, desired colors, based on features desired to be disposed on the polymeric sheath. In one or more embodiments, the step of disposing a polymeric composition onto an external surface of a polymeric sheath includes disposing a coagulant component or solution on the polymeric sheath, either by a dipping process, or by application via the rollers, before disposing a polymeric composition thereon. As discussed above, a pigment may be incorporated within the coagulant component.

Method 600 proceeds to step 606, wherein the template is engaged with a polymeric sheath, for example, the polymeric sheath discussed above. The template has the polymeric material disposed thereon. Rollers having templates, which have polymeric composition disposed thereon, are engaged with a polymeric sheath disposed on a former. The relative motion between the roller having templates and the polymeric sheath disposed on the former includes relative rotational motion and/or linear motion, while the template is in physical contact with the polymeric sheath. According to embodiments of the invention, operating parameters for the rollers are configured. Operating parameters include parameters that determine the deposition of coating solution onto the polymeric sheath. For example, the relative rotation speed of the roller(s) and the polymeric sheath, the pressure between the roller and the polymeric sheath, the relative longitudinal motion between the roller and the polymeric sheath, and the number of rotation(s) of the roller(s) while engaged with the polymeric sheath. These and other operating parameters will occur readily to those having ordinary skill on the art. At step 608, at least some of the polymeric composition disposed on the template is transferred onto the external surface of the polymeric sheath, for example, as discussed above. According to several embodiments, physical contact between the roller(s) with templates and the polymeric sheath causes transfer of at least some of the polymeric composition onto the polymeric sheath, thereby resulting in a polymeric sheath disposed with desired features comprising polymeric composition, irrespective of the former used, and without requiring operations such as embossing, printing, screen-printing, spraying, heat-staking, and the like. Accordingly, the method 600 disposes polymeric composition in shape of the desired features onto the polymeric sheath. Method 600 proceeds to step 610, at which point method 600 ends. According to embodiments of the invention, longitudinal relative motion between the template and the polymeric sheath, disposes wavelike patterns to be disposed on the polymeric sheath.

According to several embodiments, the polymeric sheath provided is a cured polymeric sheath, and in other embodiments, the polymeric sheath provided is uncured. According to embodiments of the invention, the polymeric sheath is disposed over a former. In some embodiments, the polymeric sheath is manufactured over the former, just prior to the start of method 600. Alternatively, the polymeric sheath is optionally manufactured, dried, and cured on a former before the start of method 600. Further, in some embodiments, one or more coagulant compositions are disposed onto the polymeric sheath, and in some embodiments, coagulant compositions are not deposited onto the polymeric sheath.

In at least one embodiment of the invention, a method of forming a condom comprises the steps of dipping a former into a polymeric composition, thereby forming a polymeric sheath, delivering the former having the polymeric sheath disposed thereon to apparatus for disposing additional patterns thereto, and disposing patterns comprised of an optionally colored polymeric composition thereon. In another exemplary embodiment of the invention, a pre-formed polymeric sheath is dressed onto a former, delivered to apparatus for disposing additional patterns thereto, and disposing patterns comprised of an optionally colored polymeric composition thereon.

Various embodiments described herein illustrate examples where the relative motion between the rollers and the polymeric sheath is rotational, or longitudinal. In other embodiments, relative motion between the rollers and the polymeric sheath includes relative motion along any of the three-dimensional axes (linear motion), relative rotational motion about any of the three-dimensional axes, or any combination thereof. For example, in some embodiments, the rollers oscillate (as opposed to rotate) from, for example, 10 degrees to 350 degrees around a polymeric sheath, thereby producing incomplete circumferential rings (not shown) around the polymeric sheath. While some embodiments illustrate three rollers and three tanks with polymeric composition, various embodiments are not limited thereto. According to several other embodiments, one or more rollers and one or more tanks comprising polymeric composition may be used according to desired features, as will occur readily to a person of ordinary skill in the art. Furthermore, while various embodiments have been described with respect to a condom, the embodiments disclosed herein may be used to deposit features onto other polymeric articles, including, but not limited to, gloves and the like.

While the foregoing is directed to embodiments of the invention, other embodiments of the invention may be devised without departing from the scope thereof, and the scope thereof is determined by the following claims.

What is claimed is:

1. A method for forming a polymeric article, comprising:
disposing a polymeric composition onto a template;
engaging the template with a polymeric sheath; and
transferring the polymeric composition onto an external surface of the polymeric sheath,
wherein at least one feature is disposed on the polymeric sheath by contacting the template with the polymeric sheath.

2. The method of claim 1, wherein engaging the template in physical contact with the polymeric sheath causes at least some of the polymeric composition to be transferred onto the polymeric sheath, thereby forming a feature thereon, wherein the feature is a mirror image of the template.

3. The method of claim 1, the disposing step further comprising dipping the template in the polymeric composition, wherein the template is disposed on a surface of a roller, and wherein the engaging step comprises rotating at least one of the roller or the polymeric sheath.

4. The method of claim 3, further comprising disposing additional polymeric composition on the template and increasing the duration of physical contact between the template and the polymeric sheath to increase the height of the at least one feature disposed onto the polymeric sheath.

5. The method of claim 1, wherein the engaging step further comprises at least one of dressing the polymeric sheath over a former or forming the polymeric sheath onto a former.

6. The method of claim 1, further comprising disposing a coagulant onto the polymeric sheath, wherein the coagulant is disposed before transferring the polymeric composition onto the polymeric sheath, and wherein the coagulant is deposited in a shape corresponding to the at least one feature.

7. The method of claim 1, wherein the at least one feature comprises a circumferential ring around the polymeric sheath.

8. The method of claim 1, wherein the at least one feature is disposed as a stripe along the longitudinal axis of the polymeric sheath.

9. The method of claim 1, wherein the at least one feature is disposed as a stripe in a sinusoidal pattern on the polymeric sheath.

10. The method of claim 1, wherein the at least one feature comprises at least one of a polymeric composition or coagulant solution, having a color different than a color of the polymeric sheath.

11. The method of claim 1, wherein the at least one feature comprises a first feature having only a first color and a second feature having only a second color that is different from the first color.

12. The method of claim 11, wherein the at least one feature is disposed on at least one of along a circumference of an external surface of the polymeric sheath or along a length of the polymeric sheath.
